(19) United States
(12) Patent Application Publication
(10) Pub. No.: US 2024/0115822 A1
(43) Pub. Date: Apr. 11, 2024

(54) CPAP DEVICE

(71) Applicant: Murata Manufacturing Co., Ltd., Kyoto (JP)

(72) Inventor: Keisuke INABA, Kyoto (JP)

(21) Appl. No.: 18/480,041

(22) Filed: Oct. 3, 2023

(30) Foreign Application Priority Data
Oct. 6, 2022 (JP) 2022-161644

(51) Int. Cl. A61M 16/00; A61M 16/16

(52) U.S. Cl. CPC A61M 16/0066; A61M 16/16; A61M 16/107

(57) ABSTRACT

A CPAP device includes a main unit including an inlet, an outlet, and a first electric circuit including a blower; and a base unit, to which the main unit is attachable, including an outlet, an inlet, a blower tube, and a second electric circuit different from the first electric circuit. The main unit has a lower wall, and the lower wall has multiple connection terminals connected to the first electric circuit. The base unit has an upper wall, and the upper wall has multiple connection terminals connected to the second electric circuit. The multiple connection terminals and the multiple connection terminals are disposed side by side in a direction substantially parallel to a direction in which the lower wall and the upper wall face each other. One of the multiple connection terminals and the multiple connection terminals includes a biasing force generation member that generates biasing force.

### CROSS REFERENCE TO RELATED APPLICATION

This application claims priority from Japanese Patent Application No. 2022-161644 filed on Oct. 6, 2022. The content of this application is incorporated herein by reference in its entirety.

### BACKGROUND OF THE DISCLOSURE

### 1. Field of the Disclosure

The present disclosure relates to a CPAP device in which a main unit is attachable to and detachable from a base unit.

### 2. Description of the Related Art

International Publication No. 2020/217843 describes a CPAP device including a main body unit (main unit) and a base unit. The main body unit may be used alone, but may be used in combination with the base unit.

When the main body unit and the base unit are used in combination, the main body unit is attached and fixed to the base unit.

However, with the configuration in the related art as described in International Publication No. 2020/217843, the main body unit (main unit) cannot easily be detached from the base unit. On the other hand, in a case that a mechanism for easy detachment of the main body unit from the base unit is independently provided, the device becomes large.

### BRIEF SUMMARY OF THE DISCLOSURE

It is therefore a possible benefit of the present disclosure to provide a compact CPAP device in which the main unit can easily be detached from the base unit.

A CPAP device of the present disclosure includes: a main unit including a first inlet, a first outlet, and a first electric circuit including a blower; and a base unit, to which the main unit is attachable, including a second outlet, a second inlet, a blower tube, and a second electric circuit different from the first electric circuit. The main unit has a first wall facing the base unit, and the first wall has a first terminal connected to the first electric circuit. The base unit has a second wall facing the first wall of the main unit, and the second wall has a second terminal connected to the second electric circuit. The first terminal and the second terminal are disposed side by side in a direction substantially parallel to a direction in which the first wall and the second wall face each other. One of the first terminal and the second terminal includes a biasing force generation member that generates biasing force in the direction in which the first wall and the second wall face each other.

In the configuration above, the attached state of the main unit and the base unit can be released using the biasing force and the reaction of the biasing force generated between the first terminal and the second terminal to make electrical connection.

According to the present disclosure, a compact CPAP device in which a main unit can easily be detached from a base unit may be realized.

### BRIEF DESCRIPTION OF THE SEVERAL VIEWS OF THE DRAWINGS

FIG. 1 is an external perspective view of a CPAP device according to an embodiment of the present disclosure;

FIG. 2 is an external perspective view of the CPAP device according to the embodiment of the present disclosure;

FIG. 3 is a side sectional view of the CPAP device according to the embodiment of the present disclosure;

FIG. 4A and FIG. 4B are each an external perspective view of a main unit according to the embodiment of the present disclosure;

FIG. 5A is a top view of the main unit according to the embodiment of the present disclosure, and FIG. 5B is a bottom view of the main unit;

FIG. 6A, FIG. 6B, and FIG. 6C are each a side view of the main unit according to the embodiment of the present disclosure, and FIG. 6D is a side sectional view of the main unit according to the embodiment of the present disclosure;

FIG. 7 is an external perspective view of a base unit according to the embodiment of the present disclosure;

FIG. 8A is a top view of the base unit, and FIG. 8B is a side view of the base unit;

FIG. 9A and FIG. 9B are each a side view of the base unit;

FIG. 10A and FIG. 10B are each a perspective view of a terminal electrode disposed in a base unit illustrating an example of a specific configuration;

FIG. 11A is a perspective view of a hook member, and FIG. 11B is a four side view of the hook member;

FIG. 12 is a side sectional view illustrating a popping-up state;

FIG. 13A is an enlarged sectional view of part of a side surface illustrating a fixed state, and FIG. 13B is a sectional view of each stage of part of the side surface illustrating a state that the main unit is detached from the base unit by popping-up;

FIG. 14 is a sectional view of each stage of part of the side surface illustrating a state that the main unit is detached from the base unit by popping-up; and

FIG. 15 is an enlarged sectional view of part of the side surface illustrating a state that a main unit is fixed to a base unit in a modification of the CPAP device.

### DETAILED DESCRIPTION OF THE DISCLOSURE

A CPAP device according to an embodiment of the present disclosure will be described with reference to the drawings.

### CPAP Device 10 Overview

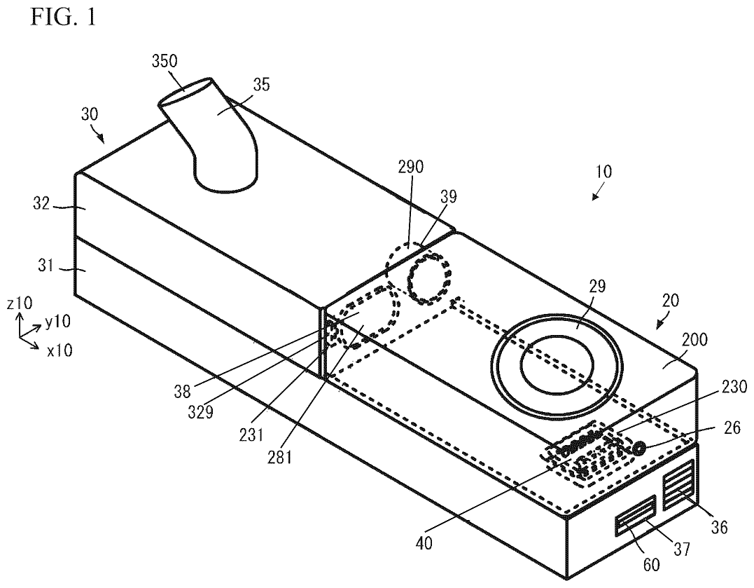

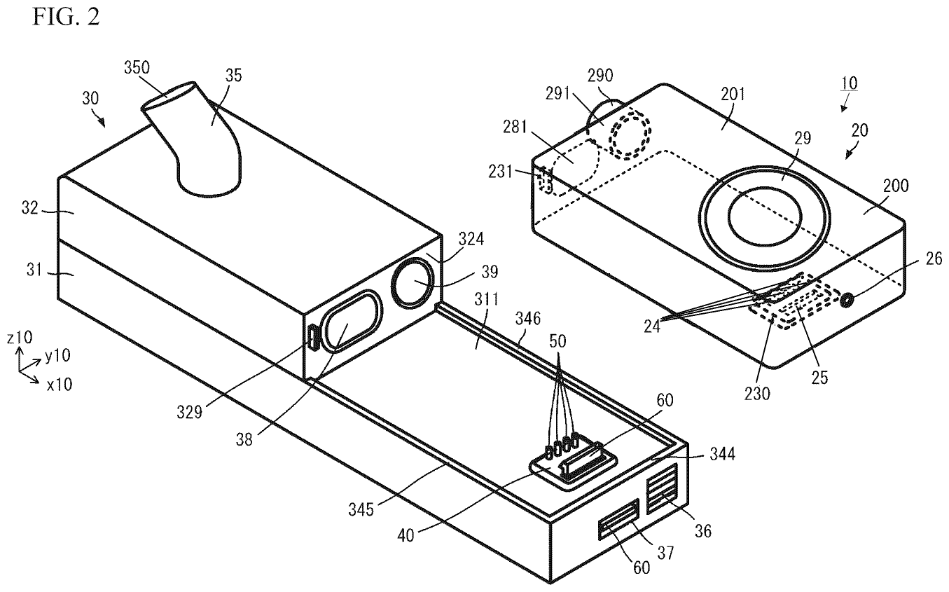

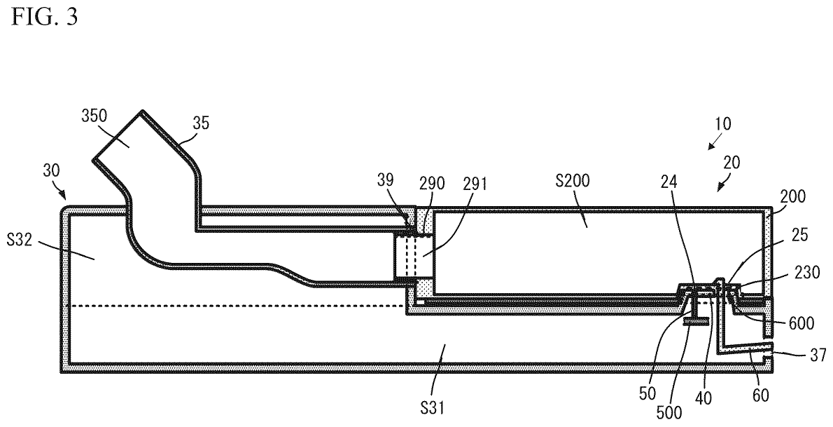

FIG. 1 and FIG. 2 are each an external perspective view of the CPAP device according to the embodiment of the present disclosure. FIG. 3 is a side sectional view of the CPAP device according to the embodiment of the present disclosure. A state in which the main unit is attached to the base unit is illustrated in FIG. 1 and FIG. 3, and a state in which the main unit is detached from the base unit is illustrated in FIG. 2.

A CPAP device 10 includes a main unit 20 and a base unit 30 as illustrated in FIG. 1, FIG. 2, and FIG. 3.

The main unit 20 has a rectangular shape having a long side direction and a short side direction in plan view. Note that the rectangular shape in the present embodiment is not limited to a perfect rectangular shape, and includes a shape with various chamfered corners. An outer shape of the main unit 20 is a rectangular parallelepiped, and a height is shorter than a length in the long side direction and a length in the short side direction. Note that the rectangular parallelepiped in the present embodiment is not limited to a perfect rectangular parallelepiped, and includes a rectangular parallelepiped having various chamfered apexes and edges.

The base unit 30 has a rectangular shape having a long side direction and a short side direction in plan view. The base unit 30 has a high height portion and a low height portion, and the high height portion is higher than the low height portion. That is, an outer shape of the base unit 30 is a shape in which a high height portion of a rectangular parallelepiped and a low height portion of a rectangular parallelepiped are connected in the long side direction of the base unit 30.

The main unit 20 may be attached to an upper surface of the low height portion of the base unit 30. In a state in which the main unit 20 is attached to the base unit 30, an outer shape of the CPAP device 10 is a rectangular parallelepiped.

In a state in which the main unit 20 is attached to the base unit 30 (attached state), a gas flow path of the main unit 20 and a gas flow path of the base unit 30 communicate with each other. Further, a first electric circuit of the main unit 20 and a second electric circuit of the base unit 30 are electrically connected to each other. Thus, the main unit 20 and the base unit 30 cooperate with each other to achieve a function of the CPAP device 10.

In a state in which the main unit 20 is detached from the base unit 30, the main unit 20 alone operates to achieve the function of the CPAP device 10.

Specific contents of an attached state, a flow path, and an electrical connection of the main unit 20 and the base unit 30 will be described later, and a specific configuration example of the main unit 20 and the base unit 30 will be described first.

### Main Unit 20

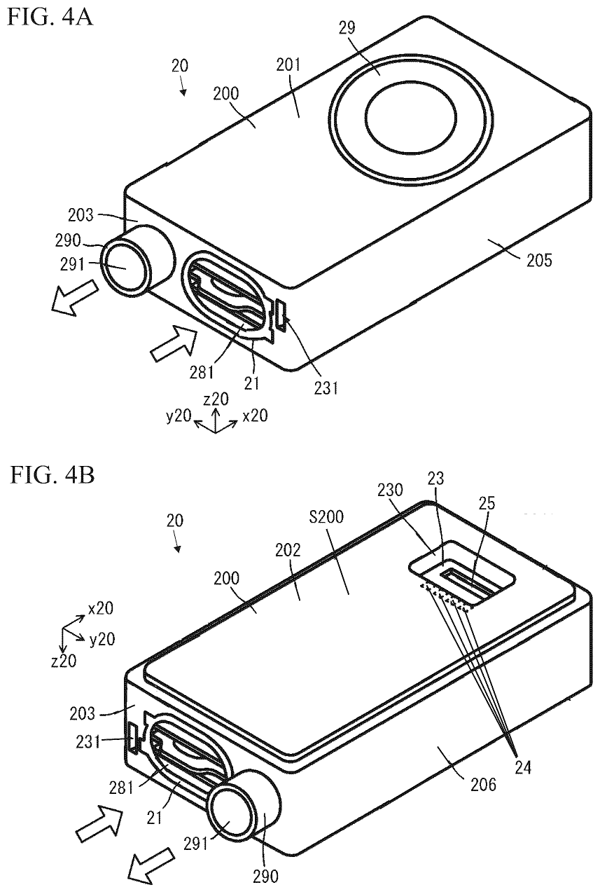

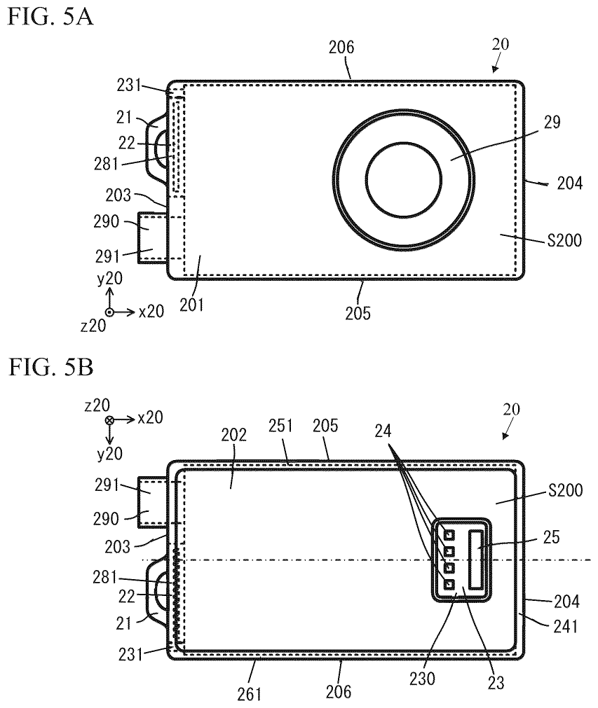

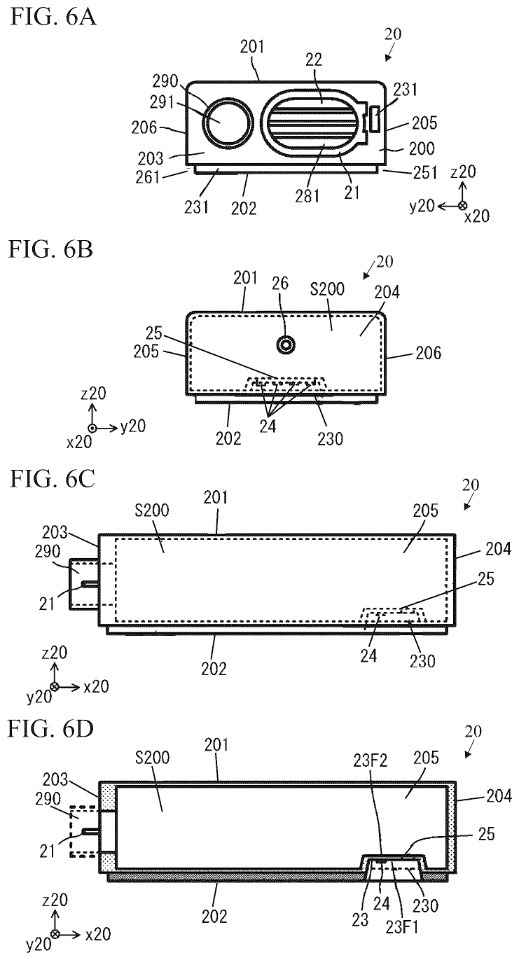

FIG. 4A and FIG. 4B are each an external perspective view of the main unit according to the embodiment of the present disclosure. FIG. 4A is a view seen from an upper surface side, and FIG. 4B is a view seen from a lower surface side. FIG. 5A is a top view of the main unit according to the embodiment of the present disclosure, and FIG. 5B is a bottom view of the main unit. FIG. 6A, FIG. 6B, and FIG. 6C are each a side view of the main unit according to the embodiment of the present disclosure, and FIG. 6D is a side sectional view of the main unit according to the embodiment of the present disclosure. FIG. 6A is a view seen from a short side wall 203 side, FIG. 6B is a view seen from a short side wall 204 side, and FIG. 6C is a view seen from a long side wall 205 side. FIG. 6D is a side sectional view seen from the same side as in FIG. 6C.

The main unit 20 includes a housing 200 as illustrated in FIG. 4A, FIG. 4B, FIG. 5A, FIG. 5B, FIG. 6A, FIG. 6B, FIG. 6C, and FIG. 6D.

An outer shape of the housing 200 is a rectangular parallelepiped having a long side direction (x20-axis direction) and a short side direction (y20-axis direction), and having a predetermined height (length in z20-axis direction). The housing 200 has an upper wall 201, a lower wall 202, a side wall 203, a side wall 204, a side wall 205, and a side wall 206.

The upper wall 201 and the lower wall 202 are opposed to each other. The side wall 203 and the side wall 204 are side walls parallel to the short side direction and are opposed to each other. The side wall 205 and the side wall 206 are side walls parallel to the long side direction and are opposed to each other.

The side wall 203, the side wall 204, the side wall 205, and the side wall 206 are each connected to peripheral portions of the upper wall 201 and the lower wall 202. Thus, the housing 200 has an inner space 5200 surrounded by the upper wall 201, the lower wall 202, the side wall 203, the side wall 204, the side wall 205, and the side wall 206. The first electric circuit (not illustrated) is disposed in the inner space 5200.

The side wall 203 has an inlet 281 and an outlet 291. The inlet 281 corresponds to a first inlet, and the outlet 291 corresponds to a first outlet. The inlet 281 and the outlet 291 each penetrate through the side wall 203 in a thickness direction. The inlet 281 and the outlet 291 allow the inner space 5200 of the housing 200 to communicate with an outer space of the housing 200. The side wall 203 is a flow path forming wall of the main unit 20, and an outer surface of the side wall 203 corresponds to a flow path forming surface of the main unit 20.

The inlet 281 and the outlet 291 are disposed side by side in a width direction of the side wall 203. The inlet 281 and the outlet 291 are disposed in this order from one end portion to the other end portion in the width direction of the side wall 203. The one end portion in the width direction of the side wall 203 is an end portion at which the side wall 203 is connected to the side wall 205, and the other end portion is an end portion at which the side wall 203 is connected to the side wall 206.

A cylindrical body 290 is provided on the side wall 203. The cylindrical body 290 has a shape protruding from the outer surface of the side wall 203. The cavity in the center of the cylindrical body 290 is connected to the outlet 291 formed on the side wall 203, and this portion also functions as the outlet 291.

An air filter 22 is disposed in the inlet 281. A filter cover 21 is provided on an outer surface side of the air filter 22. The air filter 22 is fixed to the inlet 281 being sandwiched by the filter cover 21 and the side wall 203.

A recess 231 is formed in the side wall 203. The recess 231 is formed in the vicinity of one end portion of the side wall 203. That is, the recess 231 is disposed to be separated from the cylindrical body 290 forming the outlet 291 in the width direction of the side wall 203.

A power supply port 26 is disposed on the side wall 204. The power supply port 26 is connected to the first electric circuit in the inner space 5200. Electric power is supplied to the first electric circuit by connecting the power supply port 26 to an external power supply adapter.

An operation button 29 is disposed on the upper wall 201. The operation button 29 is connected to the first electric circuit in the inner space 5200. When the operation button 29 is operated, operations such as starting and stopping of the first electric circuit are controlled.

A recess 230 is formed in the lower wall 202. The recess 230 is formed at a position closer to the other end portion than to one end portion of the lower wall 202. The one end portion of the lower wall 202 is an end portion at which the lower wall 202 is connected to the side wall 203, and the other end portion is an end portion at which the lower wall 202 is connected to the side wall 204. The recess 230 is preferably formed at a position as close as possible to the other end portion, in other words, at a position as far as possible from the cylindrical body 290 and the recess 231.

Multiple connection terminals 24 are disposed on a front surface 23F1 of a bottom wall 23 of the recess 230. The multiple connection terminals 24 are disposed side by side in parallel with a short side direction of the lower wall 202. The multiple connection terminals 24 are film-like electrode patterns (rectangular electrode patterns in plan view, for example) and are connected to the first electric circuit.

A groove 25 is formed in the bottom wall 23. The groove 25 penetrates through the bottom wall 23 and has a shape extending parallel to the short side direction of the lower wall 202. The groove 25 is formed at a position closer to the other end portion of the lower wall 202 than the multiple connection terminals 24.

### Base Unit 30

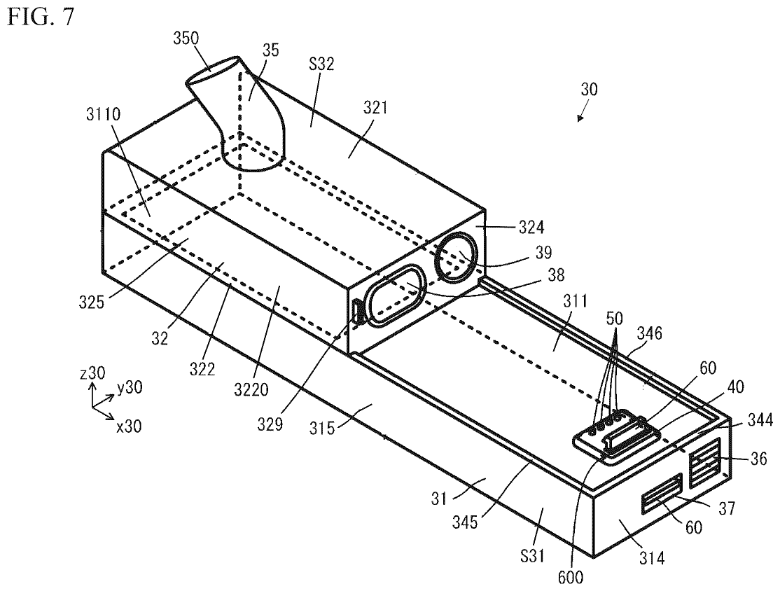

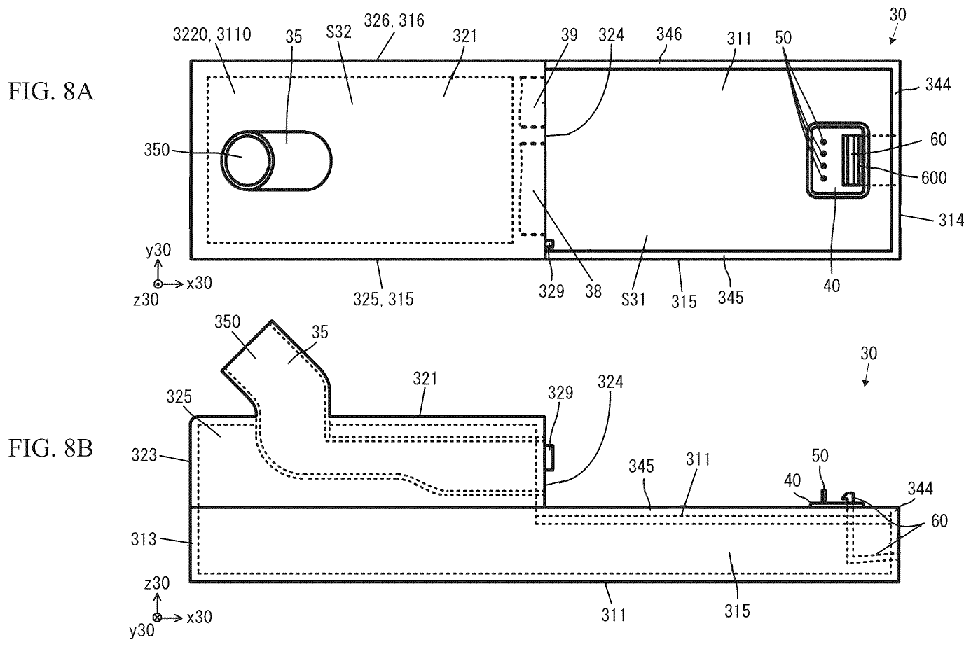

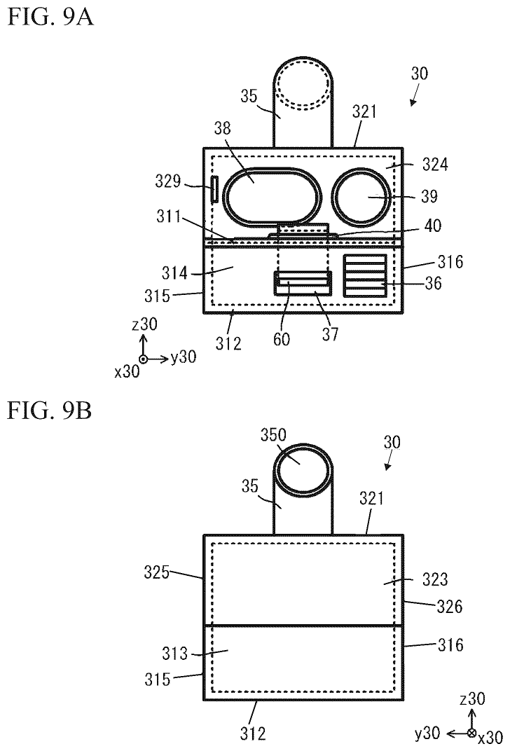

FIG. 7 is an external perspective view of the base unit according to the embodiment of the present disclosure. FIG. 8A is a top view of the base unit, and FIG. 8B, FIG. 9A, and FIG. 9B are each a side view of the base unit. FIG. 8B is a view seen from a long side wall 315 side and a long side wall 325 side, FIG. 9A is a view seen from a short side wall 314 side and a short side wall 324 side, and FIG. 9B is a view seen from a short side wall 313 side and a short side wall 323 side.

The base unit 30 includes multiple housings 31 and 32 as illustrated in FIG. 7, FIG. 8A, FIG. 8B, FIG. 9A, and FIG. 9B.

### Base Unit 30 Outline Shape

An outer shape of each of the housing 31 and the housing 32 is a rectangular parallelepiped having a long side direction (x30-axis direction) and a short side direction (y30-axis direction), and having a predetermined height (length in z30-axis direction). A length of the housing 31 in the long side direction is longer than a length of the housing 32 in the long side direction.

The housing 32 is disposed on an upper wall 311 side of the housing 31, and the housing 31 and the housing 32 are connected to each other. The inner space S31 of the housing 31 and the inner space S32 of the housing 32 communicate with each other through an opening 3110 of the housing 31 and an opening 3220 of the housing 32.

A side wall 313 of the housing 31 and a side wall 323 of the housing 32 are flush with each other, a side wall 315 of the housing 31 and a side wall 325 of the housing 32 are flush with each other, and a side wall 316 of the housing 31 and a side wall 326 of the housing 32 are flush with each other.

Thus, the base unit 30 has a step at an intermediate position in the long side direction, and has a high height portion where the housing 31 and the housing 32 overlap with each other and a low height portion where only the housing 31 is provided.

### Housing 31

The housing 31 has an upper wall 311, a lower wall 312, the side wall 313, a side wall 314, the side wall 315, and the side wall 316. The upper wall 311 and the lower wall 312 are opposed to each other. The side wall 313 and the side wall 314 are side walls parallel to the short side direction and are opposed to each other. The side wall 315 and the side wall 316 are side walls parallel to the long side direction and are opposed to each other. The upper wall 311 corresponds to a second wall of the present disclosure.

The side wall 313, the side wall 314, the side wall 315, and the side wall 316 are each connected to peripheral portions of the upper wall 311 and the lower wall 312. Thus, the housing 31 has an inner space S31 surrounded by the upper wall 311, the lower wall 312, the side wall 313, the side wall 314, the side wall 315, and the side wall 316. At least part of the second electric circuit is disposed in the inner space S31 (not illustrated).

The opening 3110 is formed in the upper wall 311. The opening 3110 is a hole penetrating through the upper wall 311 in a thickness direction. The opening 3110 has a predetermined length in the long side direction and is open over substantially the entire upper wall 311 in the short side direction. The opening 3110 is formed so as to leave a predetermined area of the upper wall 311 on a side connected to the side wall 314. In other words, the opening 3110 is formed in a region inside a predetermined width relative to a region in which the housing 32 is disposed.

Frames 344, 345, and 346 are formed on the peripheral portion of the upper wall 311. The frames 344, 345, and 346 protrude upward from an upper surface of the upper wall 311. The frame 344 extends along a connection portion of the upper wall 311 and the side wall 314, the frame 345 extends along a connection portion of the upper wall 311 and the side wall 315, and the frame 346 extends along a connection portion of the upper wall 311 and the side wall 316.

A protrusion 40 is formed on the upper wall 311. The protrusion 40 is formed on the upper wall 311 at a position close to the side wall 314. More specifically, the protrusion 40 is formed at a position to be fitted into the recess 230 of the main unit 20, in a state in which the main unit 20 is attached to the base unit 30.

Multiple connection terminals 50 are disposed on a top wall of the protrusion 40. The multiple connection terminals 50 are disposed side by side in parallel with a short side direction of the upper wall 311. The multiple connection terminals 50 are connected to the second electric circuit.

In the CPAP device 10 in a state in which the main unit 20 is attached to the base unit 30, the multiple connection terminals 50 are disposed at positions overlapping with the multiple connection terminals 24 when viewed from an upper surface side. The multiple connection terminals 50 are rod bodies extending in a direction orthogonal to a front surface of the top wall of the protrusion 40. The multiple connection terminals 50 correspond to pin terminals of the present disclosure.

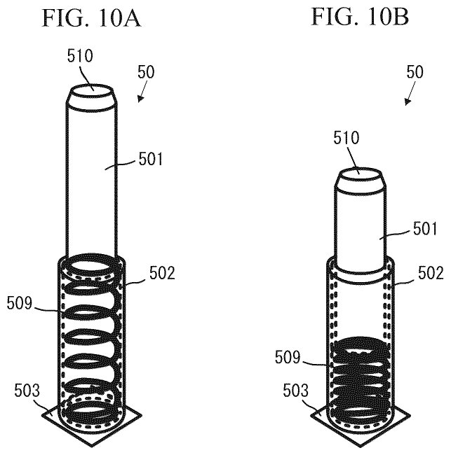

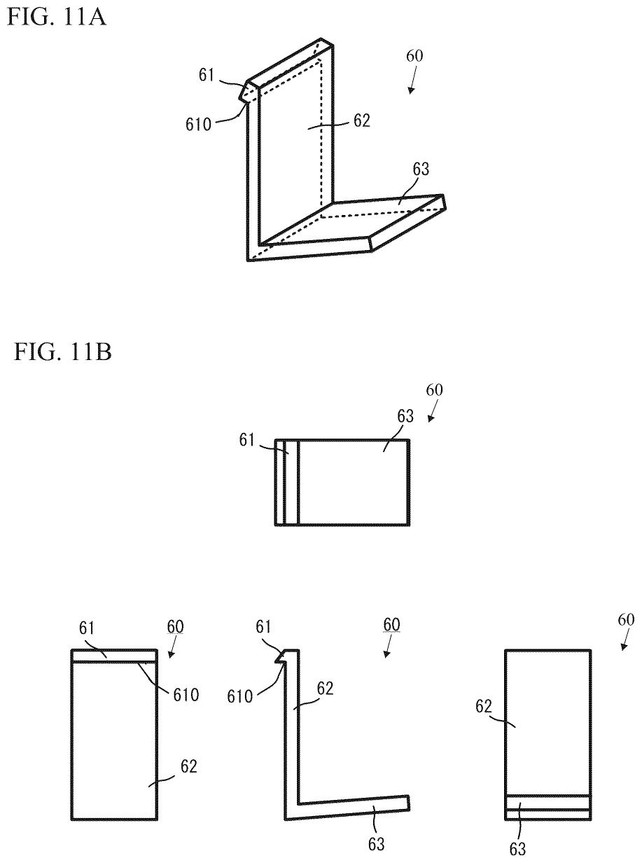

FIG. 10A and FIG. 10B are each a perspective view of a terminal electrode disposed in the base unit illustrating an example of a specific configuration. Note that a spring is indicated by a solid line in FIG. 10A and FIG. 10B to facilitate viewing, but the spring is accommodated inside a housing of the terminal electrode.

The connection terminal 50 includes a columnar plunger 501, a cylindrical housing 502, a bottom wall 503, and a spring 509. The plunger 501 is accommodated in the housing 502 so as to be movable in an extending direction of the housing 502 (axial direction of cylindrical shape). The bottom wall 503 closes one end of the housing 502.

The spring 509 is disposed in the housing 502. One end of the spring 509 is in contact with the bottom wall 503, and the other end of the spring 509 is in contact with a bottom surface of the plunger 501.

With the configuration above, the connection terminal 50 can expand and contract in the extending direction as illustrated in FIG. 10A and FIG. 10B, and can generate biasing force in the extending direction.

A groove 600 is formed in the top wall of the protrusion 40. The groove 600 penetrates through the top wall of the protrusion 40 and has a shape extending parallel to the short side direction of the upper wall 311. The groove 600 is formed on the upper wall 311 at a position closer to the side wall 314 than the multiple connection terminals 50.

A circuit substrate 500 constituting the second electric circuit is disposed in the inner space S31 of the housing 31. The multiple connection terminals 50 each penetrate through the top wall of the protrusion 40, and are attached and fixed to the circuit substrate 500.

A hook member 60 is disposed in the housing 31. FIG. 11A is a perspective view of the hook member, and FIG. 11B is a four side view of the hook member.

The hook member 60 has a head 61, a support body 62, and an operation body 63 as illustrated in FIG. 11A and FIG. 11B. The support body 62 and the operation body 63 are each a flat plate. The operation body 63 is connected to one end of the support body 62. The main surface of the operation body 63 and the main surface of the support body 62 form an angle of approximately 90°.

The head 61 is connected to the other end of the support body 62. The head 61 has a shape protruding toward a side of the support body 62 opposite to a side to which the operation body 63 extends. An inner hook surface 610 of the head 61 connected to the support body 62 is substantially perpendicular to a direction in which the support body 62 extends.

The support body 62 of the hook member 60 is inserted into the groove 600. The head 61 and part of the support body 62 on a head 61 side protrude outward from the top wall of the protrusion 40. The other portion of the support body 62 and the operation body 63 are accommodated in the inner space S31. The tip end portion of the operation body 63 is disposed at a position operable from the outside through an opening 37.

### Housing 32

The housing 32 has an upper wall 321, the side wall 323, a side wall 324, the side wall 325, and the side wall 326. The side wall 323 and the side wall 324 are side walls parallel to the short side direction and are opposed to each other. The side wall 325 and the side wall 326 are side walls parallel to the long side direction and are opposed to each other.

The side wall 323, the side wall 324, the side wall 325, and the side wall 326 are each connected to a peripheral portion of the upper wall 321. Thus, the housing 32 has an inner space S32 surrounded by the upper wall 321, the side wall 323, the side wall 324, the side wall 325, and the side wall 326.

An opening 3220 is formed on the housing 32 on a side opposed to the upper wall 321.

A blower tube 35 is formed on the upper wall 321. The blower tube 35 has a shape protruding outward and toward a side wall 323 side from the upper wall 321. An opening 350 of the blower tube 35 serves as a final air blowing port for supplying desired air (wind) to a user in a state in which the main unit 20 is attached to the base unit 30.

The side wall 324 is a wall having a surface that forms a step of the base unit 30. The side wall 324 has an outlet 38 and an inlet 39. The outlet 38 corresponds to the second outlet, and the inlet 39 corresponds to the second inlet. The outlet 38 and the inlet 39 each penetrate through the side wall 324 in a thickness direction. The outlet 38 and the inlet 39 allow the inner space S32 of the housing 32 to communicate with an outer space of the housing 32. The side wall 324 is a flow path forming wall on a side of the base unit 30, and an outer surface of the side wall 324 corresponds to a flow path forming surface of the base unit 30.

The outlet 38 and the inlet 39 are disposed side by side in a width direction of the side wall 324. The outlet 38 and the inlet 39 are disposed in this order from one end portion to the other end portion in the width direction of the side wall 324. The one end portion in the width direction of the side wall 324 is an end portion at which the side wall 324 is connected to the side wall 325, and the other end portion is an end portion at which the side wall 324 is connected to the side wall 326.

A protrusion 329 is formed on the side wall 324. The protrusion 329 is formed in the vicinity of the one end portion of the side wall 324. That is, the protrusion 329 is disposed to be separated from the inlet 39 in the width direction of the side wall 324.

### CPAP Device 10 Operation Overview

### Use of Main Unit 20 Alone

The main unit 20 includes a blower (not illustrated) electrically connected to the first electric circuit. The blower is disposed in the inner space 5200 of the housing 200. When the blower is driven, the main unit 20 sucks air from the inlet 281 and discharges air from the outlet 291. Being provided with the air filter 22 in the inlet 281, the main unit 20 supplies gas (air) having a desired degree of cleanliness to a user from the outlet 291.

### Use of Main Unit 20 Attached to Base Unit 30

When the main unit 20 is attached to the base unit 30, the main unit 20 is disposed on an upper surface (upper surface of upper wall 311) of the low height portion of the base unit 30. The lower wall 202 of the housing 200 of the main unit 20 and the upper wall 311 of the housing 31 of the base unit 30 face each other.

Further, the outer surface of the side wall 203 of the housing 200 and the outer surface of the side wall 324 of the housing 32 come close to each other or come into contact with each other to be in a close contact state. Note that the side wall 203 and the side wall 324 may be brought into close contact with each other via an elastic body.

At this time, the cylindrical body 290 constituting the outlet 291 of the main unit 20 is inserted into the inlet 39 of the base unit 30. Further, the inlet 281 of the main unit 20 and the outlet 38 of the base unit 30 overlap with each other when viewed from an x-axis (x10-axis). As a result, a flow path forming surface on a side of the main unit 20 and a flow path forming surface on the side of the base unit 30 closely face each other. The inlet 281 of the main unit 20 and the outlet 38 of the base unit 30 are inserted and fitted to each other without causing gas leakage. The outlet 291 of the main unit 20 and the inlet 39 of the base unit 30 are inserted and fitted to each other without causing gas leakage.

When the blower is driven in the state above, the base unit 30 sucks gas from an inlet 36 and discharges the gas from the outlet 38 through the inner space S31 and the inner space S32. The main unit 20 sucks the gas discharged from the outlet 38 through the inlet 281 and discharges the gas from the outlet 291. The base unit 30 sucks the gas discharged from the outlet 291 through the inlet 39 and discharges the gas from the opening 350 of the blower tube 35. Thus, the CPAP device 10 in a state in which the main unit 20 is attached to the base unit 30 supplies gas (air) having a desired degree of cleanliness to a user from the opening 350 of the blower tube 35.

Although not illustrated in the drawings, a water storage tank is accommodated in the high height portion of the base unit 30. Further, the base unit 30 is provided with a heater for heating water in the water storage tank.

The base unit 30 has an inlet to take water vapor heated by the heater in a flow path leading from the inlet 39 to the opening 350 of the blower tube 35. With this, the CPAP device 10 in a state in which the main unit 20 is attached to the base unit 30 supplies gas (air) having desired humidity to a user from the opening 350 of the blower tube 35.

At this time, the protrusion 40 of the base unit 30 is inserted into the recess 230 of the main unit 20. Then, a top surface 510 of each of the multiple connection terminals 50 of the base unit 30 is in contact with and electrically connected to each of the multiple connection terminals 24 of the main unit 20.

At this time, the multiple connection terminals 50 are pushed in by the main unit 20 (multiple connection terminals 24). With this, the multiple connection terminals 50 generate the biasing force in a direction (electrical connection direction) to connect to the multiple connection terminals 24. As a result, the connection resistance between the multiple connection terminals 50 and the multiple connection terminals 24 may be lowered, and the reliability of electrical connection is increased.

Further, the hook member 60 of the base unit 30 is inserted into the groove 25 of the housing 200 of the main unit 20. The head 61 of the hook member 60 reaches the inner space 5200 of the housing 200, and the hook surface 610 of the head 61 comes into contact with a rear surface 23F2 of the bottom wall 23 of the recess 230. Thus, the head 61 is engaged with the groove 25 of the bottom wall 23. As a result, the main unit 20 is fixed to the base unit 30.

Further, the protrusion 329 of the base unit 30 is inserted into the recess 231 of the main unit 20, and the cylindrical body 290 of the main unit 20 is inserted into the inlet 39 of the base unit 30. With this, the fixed state of the main unit 20 to the base unit 30 is further stabilized.

Further, the frames 344, 345, and 346 of the base unit 30 are respectively fitted into grooves 241, 251, and 261 of the main unit 20. With this, the main unit 20 is more firmly fixed to the base unit 30.

Further, when the main unit 20 and the base unit 30 are connected to each other, a condition (sealing property, for example) required for a gas flow path connection is different from a condition (stability of electrical connection, for example) required for an electrical connection. With the main unit 20 and the base unit 30 in the CPAP device 10, a set of surfaces (walls) for the gas flow path connection and a set of surfaces (walls) for the electrical connection are different sets of surfaces (walls). With this, in the CPAP device 10, the condition required for the gas flow path connection and the condition required for the electrical connection may be achieved by different sets of surfaces (walls). Therefore, in the CPAP device 10, it is possible to easily realize a configuration that simultaneously satisfies the condition required for the gas flow path connection and the condition required for the electrical connection.

As described above, the gas flow path of the main unit 20 and the gas flow path of the base unit 30 communicate with each other ensuring sealing property, and the first electric circuit of the main unit 20 and the second electric circuit of the base unit 30 are electrically connected with high reliability.

### Pop-Up Mechanism

Detaching Main Unit 20 from Base Unit 30

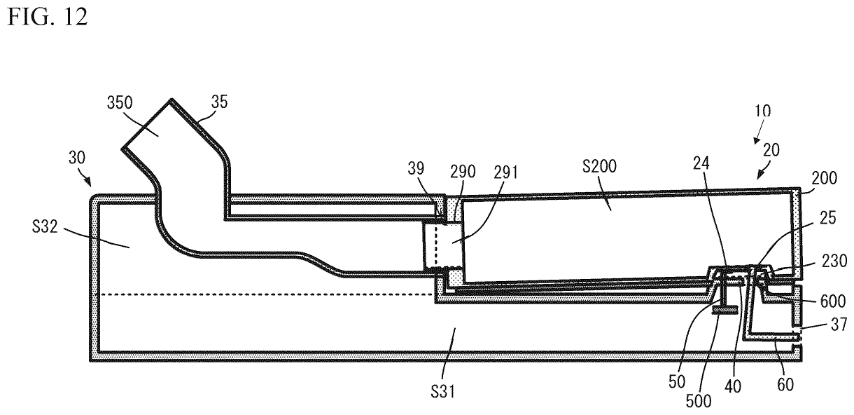

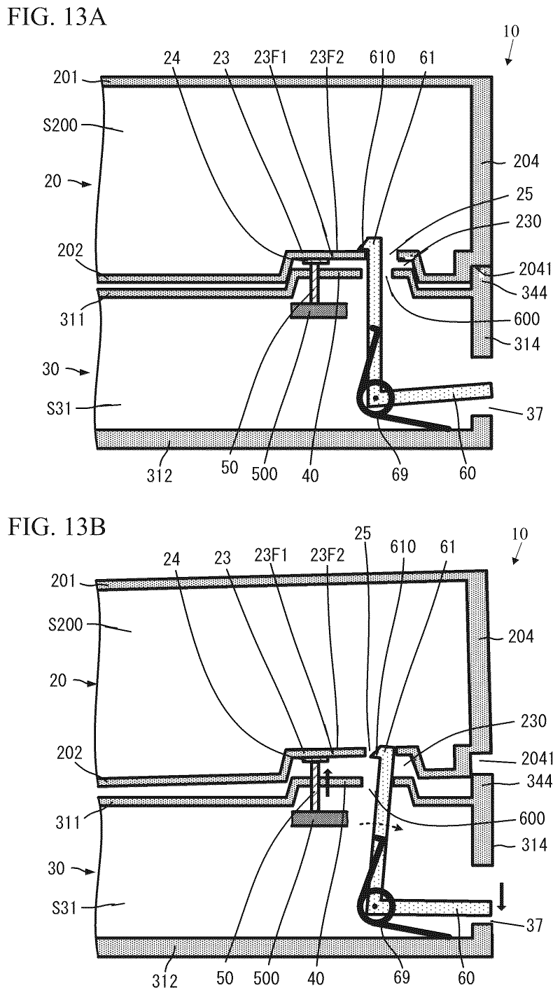

FIG. 12 is a side sectional view illustrating a popping-up state. FIG. 13A is an enlarged sectional view of part of a side surface illustrating a fixed state, and FIG. 13B is a sectional view of each stage of part of the side surface illustrating a state in which the main unit is detached from the base unit by popping-up.

In a state in which the main unit 20 is attached and fixed to the base unit 30 as illustrated in FIG. 13A, the hook surface 610 of the head 61 of the hook member 60 comes into contact with the rear surface 23F2 of the bottom wall 23 of the recess 230. With this, the head 61 presses the bottom wall 23 toward the upper wall 311 side of the base unit 30, and the multiple connection terminals 24 press the multiple connection terminals 50. Thus, the multiple connection terminals 50 are each made short in the axial direction, and biasing force to the multiple connection terminals 24 by the spring 509 is generated.

Meanwhile, pressing force is applied to the rear surface 23F2 of the bottom wall 23 from the head 61 of the hook member 60. The hook member 60 is fixed to the housing 31 of the base unit 30. As a result, even when the biasing force of the multiple connection terminals 50 is applied to the multiple connection terminals 24 and the bottom wall 23, the main unit 20 is fixed to the base unit 30 without being separated.

When the operation body 63 of the hook member 60 is pressed toward a lower wall 312 side, the hook member 60 rotates with the connection portion of the support body 62 and the operation body 63 as an axis, as illustrated in FIG. 13B. As a result, the head 61 is separated from the rear surface 23F2 of the bottom wall 23 and moves into the groove 25.

With the above-described motion of the head 61, the pressing force to the rear surface 23F2 of the bottom wall 23 is released, and only the biasing force from the multiple connection terminals 50 is applied to the bottom wall 23. As a result, the main unit 20 is pushed upward from the upper wall 311 of the housing 31 of the base unit 30. In other words, the main unit 20 is popped-up from the upper wall 311 of the housing 31 of the base unit 30.

As described above, in the CPAP device 10, the main unit 20 may easily be detached from the base unit 30 with the biasing force of the multiple connection terminals 50 for electrical connection. Thus, the CPAP device 10 does not need to have an additive separate member for generating the biasing force, and may be reduced in size.

Further, as described above, since the cylindrical body 290 of the housing 200 is inserted into the inlet 39 of the housing 32 and the protrusion 329 of the housing 32 is inserted into the recess 231 of the housing 200, these portions serve as fulcrums, and in the main unit 20, only a side wall 204 side portion of the housing 200 is pushed upward.

The side wall 203 and the side wall 204 are walls at both ends in the long side direction of the housing 200. Therefore, the distance between the fulcrums and the point of force when the main unit 20 is pushed up can be elongated. Thus, the main unit 20 can be pushed up with smaller force. That is, in the CPAP device 10, the main unit 20 can be popped-up from the base unit 30 only by the biasing force of the multiple connection terminals 50 used for electrical connection without additively providing a mechanism having large biasing force.

Thus, the CPAP device 10 may be reduced in size while enabling the main unit 20 to be easily detached from the base unit 30.

Further, in the CPAP device 10, the hook member 60 constantly applies force in a direction toward the side wall 203 by a biasing force generation member 69 for the hook member, such as a coil spring. Thus, in a state in which the main unit 20 is attached to the base unit 30, force is applied not only in a direction in which the multiple connection terminals 24 push the multiple connection terminals 50, but also in a direction in which the main unit 20 is pushed toward the side wall 324 of the base unit 30. With this, the sealing property in the flow path communication between the main unit 20 and the base unit 30 may further be improved in the CPAP device 10.

### Attach Main Unit 20 to Base Unit 30

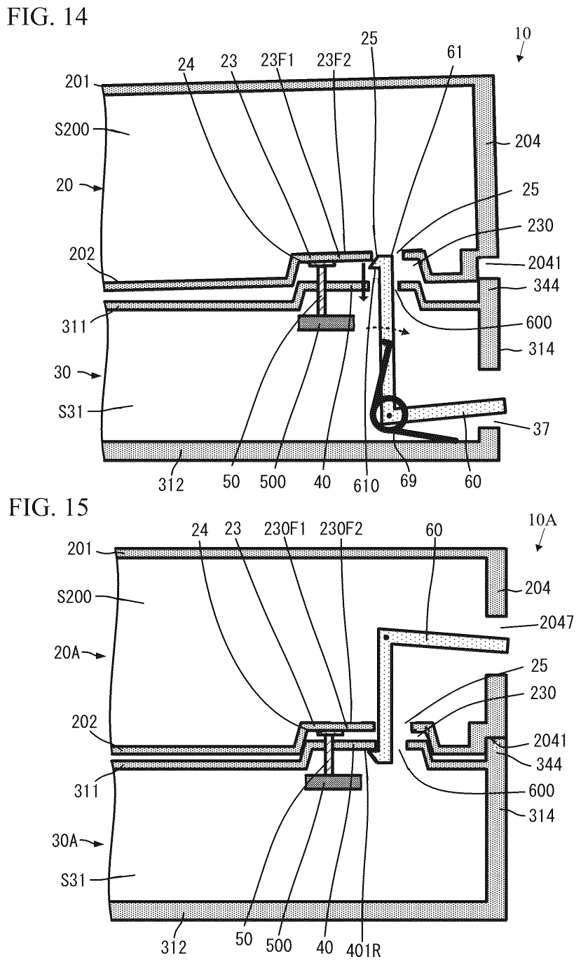

FIG. 14 is a sectional view of each stage of part of the side surface illustrating a state in which the main unit is detached from the base unit by popping-up.

When the main unit 20 is attached to the base unit 30, the main unit 20 is brought close to the upper wall 311 of the housing 31 of the base unit 30 in a state in which the cylindrical body 290 of the housing 200 is inserted into the inlet 39 of the housing 32 and the protrusion 329 of the housing 32 is inserted into the recess 231 of the housing 200. When the main unit 20 approaches the upper wall 311, the bottom wall 23 comes into contact with the head 61 of the hook member 60 and applies pressing force. The hook member 60 is rotated with the pressing force from the bottom wall 23, and the head 61 is temporarily moved to a groove 25 side.

Then, when the bottom wall 23 rotates to a position where the head 61 does not overlap with the bottom wall 23 in plan view, the bottom wall 23 is further pushed toward a housing 31 side, and the multiple connection terminals 24 push the multiple connection terminals 50. When the hook surface 610 of the head 61 reaches above the rear surface 23F2 of the bottom wall 23, the head 61 receives the biasing force from the biasing force generation member 69 for the hook member and rotates toward the side wall 203 side. Then, the hook surface 610 of the head 61 comes into contact with the rear surface 23F2 of the bottom wall 23, and the main unit 20 is attached and fixed to the base unit 30.

As described above, in the CPAP device 10, when the main unit 20 is attached to the base unit 30 as well, the main unit 20 may easily be attached and fixed to the base unit 30.

The switch of the present application is realized by the biasing force of the multiple connection terminals 50 described above and the structure in which the hook member 60 engages with and separates from the groove 25 of the bottom wall 23.

### Modification

FIG. 15 is an enlarged sectional view of part of a side surface illustrating a state in which a main unit is fixed to a base unit in a CPAP device according to a modification.

As illustrated in FIG. 15, a CPAP device 10A according to the modification is different from the CPAP device 10 described above in that the hook member 60 is provided not in a base unit 30A but in a main unit 20A. Other configurations of the CPAP device 10A are the same as those of the CPAP device 10, and a description of the same portions is omitted.

The CPAP device 10A includes the main unit 20A and the base unit 30A. The main unit 20A includes the hook member 60. In a state in which the main unit 20A is attached to the base unit 30A, the support body 62 of the hook member 60 is inserted through the groove 25 and the groove 600, and the head 61 comes into contact with a rear surface 401R of the protrusion 40.

Even with the configuration above, the CPAP device 10A may achieve the same operational effects as the CPAP device 10 does.
<1> A CPAP device, comprising: a main unit including a first inlet, a first outlet, and a first electric circuit including a blower; and a base unit, to which the main unit is attachable, including a second outlet, a second inlet, a blower tube, and a second electric circuit different from the first electric circuit, wherein the main unit has a first wall facing the base unit, and the first wall has a first terminal connected to the first electric circuit, the base unit has a second wall facing the first wall of the main unit, and the second wall has a second terminal connected to the second electric circuit, the first terminal and the second terminal are disposed side by side in a direction substantially parallel to a direction in which the first wall and the second wall face each other, and one of the first terminal and the second terminal includes a biasing force generation member to generate biasing force in the direction in which the first wall and the second wall face each other.
<2> The CPAP device according to <1>, wherein one of the main unit and the base unit includes a switch to change over between an attached state in which the main unit is attached to the base unit and a released state in which the attached state is released with the biasing force.
<3> The CPAP device according to <2>, wherein the switch includes a groove formed in one of the main unit and the base unit and a hook member disposed on the other of the main unit and the base unit, and the attached state and the released state are changed over depending on a locking state of the hook member to the groove.
<4> The CPAP device according to any one of <1> to <3>, wherein one of the first terminal and the second terminal is a pin terminal to generate the biasing force in the direction in which the first wall and the second wall face each other, and the other of the first terminal and the second terminal is a film-like electrode pattern.
<5> The CPAP device according to <2> or <3>, wherein the main unit has a flow path forming wall in which the first outlet is formed and a gas flow path on a side of the main unit is formed, and the first wall having the first terminal on different surfaces, the base unit has a flow path forming wall in which the second inlet is formed and a gas flow path on a side of the base unit is formed, and an outer surface of the second wall having the second terminal on different surfaces, and the main unit is attached to the base unit in a state in which the flow path forming wall on the side of the main unit and the flow path forming wall on the side of the base unit face each other, and the first wall on the side of the main unit and the second wall on the side of the base unit face each other.
<6> The CPAP device according to <5>, wherein the base unit includes a high height portion and a low height portion, the high height portion and the low height portion are physically connected to each other, a side surface formed by a step of the high height portion and the low height portion is an outer surface of the flow path forming wall on the side of the base unit, and an upper surface of the low height portion is an outer surface of the second wall.
<7> The CPAP device according to <6>, wherein the first outlet is a cylindrical body protruding from a wall surface of the main unit to the outside, and in the attached state, the cylindrical body is inserted into the second inlet.
<8> The CPAP device according to <7>, wherein a flow path forming surface on the side of the base unit and a flow path forming surface on the side of the main unit are provided with a protrusion and a recess which are inserted and fitted to each other.
<9> The CPAP device according to <8>, wherein a formation position of the second inlet is in the vicinity of one end in a width direction of the side surface formed by the step, and a formation position of the protrusion or the recess formed on the side surface formed by the step is in the vicinity of the other end in the width direction of the side surface formed by the step.
<10> The CPAP device according to any one of <6> to <9>, wherein the first terminal is disposed at a position that is in the vicinity of a surface opposed to a flow path forming surface on the side of the main unit and that is in a lower surface of the main unit facing the upper surface of the low height portion, and the second terminal is disposed in the vicinity of a side surface of the low height portion that is most distant from the side surface formed by the step on the upper surface of the low height portion.
<11> The CPAP device according to <10>, wherein the upper surface of the low height portion is provided with a frame having a shape along an outer periphery of the upper surface, the main unit has a groove along an outer periphery of the lower surface, and the frame is inserted into the groove.

What is claimed is:

1. A CPAP device, comprising:
a main unit including a first inlet, a first outlet, and a first electric circuit including a blower; and
a base unit, to which the main unit is attachable, including a second outlet, a second inlet, a blower tube, and a second electric circuit different from the first electric circuit,
wherein the main unit has a first wall facing the base unit, and the first wall has a first terminal connected to the first electric circuit,
the base unit has a second wall facing the first wall of the main unit, and the second wall has a second terminal connected to the second electric circuit,
the first terminal and the second terminal are disposed side by side in a direction substantially parallel to a direction in which the first wall and the second wall face each other, and
one of the first terminal and the second terminal includes a biasing force generation member configured to generate biasing force in the direction in which the first wall and the second wall face each other.

2. The CPAP device according to claim 1,
wherein one of the main unit and the base unit includes
a switch configured to change over between an attached state in which the main unit is attached to the base unit and a released state in which the attached state is released with the biasing force.

3. The CPAP device according to claim 2,
wherein the switch includes
a groove provided in one of the main unit and the base unit and
a hook member disposed on another of the main unit and the base unit, and
the attached state and the released state are changed over depending on a locking state of the hook member to the groove.

4. The CPAP device according to claim 1,
wherein one of the first terminal and the second terminal is a pin terminal configured to generate the biasing force in the direction in which the first wall and the second wall face each other, and
another of the first terminal and the second terminal is a film-like electrode pattern.

5. The CPAP device according to claim 2,
wherein the main unit has
a flow path forming wall, in which the first outlet is provided and a gas flow path on a side of the main unit is provided, and the first wall having the first terminal on different surfaces,
the base unit has
a flow path forming wall, in which the second inlet is provided and a gas flow path on a side of the base unit is provided, and an outer surface of the second wall having the second terminal on different surfaces, and
the main unit is attached to the base unit in a state in which the flow path forming wall on the side of the main unit and the flow path forming wall on the side of the base unit face each other, and the first wall on the side of the main unit and the second wall on the side of the base unit face each other.

6. The CPAP device according to claim 5,
wherein the base unit includes a high height portion and a low height portion,
the high height portion and the low height portion are physically connected to each other,
a side surface comprising a step of the high height portion and the low height portion is an outer surface of the flow path forming wall on the side of the base unit, and
an upper surface of the low height portion is an outer surface of the second wall.

7. The CPAP device according to claim 6,
wherein the first outlet is a cylindrical body protruding from a wall surface of the main unit to an outside, and
in the attached state, the cylindrical body is inserted into the second inlet.

8. The CPAP device according to claim 7,
wherein a flow path forming surface on the side of the base unit and a flow path forming surface on the side of the main unit are provided with a protrusion and a recess inserted and fitted to each other.

9. The CPAP device according to claim 8,
wherein a formation position of the second inlet is in a vicinity of one end in a width direction of the side surface comprising the step, and
a formation position of the protrusion or the recess provided on the side surface comprising the step is in a vicinity of another end in the width direction of the side surface comprising the step.

10. The CPAP device according to claim 6,
wherein the first terminal is disposed at a position being in a vicinity of a surface opposed to a flow path forming surface on the side of the main unit and being in a lower surface of the main unit facing the upper surface of the low height portion, and
the second terminal is disposed in a vicinity of a side surface of the low height portion being most distant from the side surface comprising the step on the upper surface of the low height portion.

11. The CPAP device according to claim 10,
wherein the upper surface of the low height portion is provided with a frame having a shape along an outer periphery of the upper surface,
the main unit has a groove along an outer periphery of the lower surface, and
the frame is inserted into the groove.
